(19) United States
(12) Patent Application Publication
(10) Pub. No.: US 2025/0041117 A1
(43) Pub. Date: Feb. 6, 2025

(54) ANTI-VACUUM SURGE MODULE WITH RECTANGULAR VALVE CHAMBER

(71) Applicant: Johnson & Johnson Surgical Vision, Inc., Irvine, CA (US)

(72) Inventors: Andres Claudio Altmann, Haifa (IL); Ilya Sitnitsky, Nahariya (IL); Assaf Govari, Haifa (IL); Elad Avraham Diukman, Haifa (IL); Sergey Sobol, Haifa (IL); Alexander Shechtman, Haira (IL); Amit Fuchs, Hogla (IL)

(21) Appl. No.: 18/785,559

(22) Filed: Jul. 26, 2024

Related U.S. Application Data
(60) Provisional application No. 63/530,401, filed on Aug. 2, 2023.

(51) Int. Cl. A61F 9/007; F16K 3/26; F16K 31/08; F16K 37/00

(52) U.S. Cl. CPC A61F 9/00745; F16K 3/26; F16K 31/08; F16K 37/005

(57) ABSTRACT

An anti-vacuum surge (AVS) cartridge includes a solenoid valve with a magnetic plunger having parallel flattened surfaces, in a valve cavity of a rectangular or other similar cross section. The flattened surfaces of the plunger are oppositely disposed from each other and are in line with the flow path through the aspiration channel of the AVS cartridge.

### CROSS-REFERENCE TO RELATED APPLICATION

This application claims the benefit of U.S. Provisional Patent Application 63/530,401, filed Aug. 2, 2023, which is incorporated herein by reference.

### TECHNICAL FIELD

The present disclosure relates generally to connector modules for phacoemulsification handles or hand pieces, and particularly to valves for controlling the flow of fluid due to vacuum suction through a line in the handpiece to prevent anti-vacuum surges.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses an ultrasonic handpiece, also known as a phacoemulsification probe, which has a handle and a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

The handpiece is provided its power from a connector module, which communicates with a console. The connector module connects electrically to the handpiece, through multiple individual module connections. Through these multiple individual module connections, power and data are transferred, to operate the connector module and the handpiece.

### SUMMARY

An ophthalmic fluid dynamics system comprises a solenoid valve. The solenoid valve comprises: a valve body, a solenoid coil, a plunger, and a controller. The valve body comprises ports including an inlet port and an outlet port, and a valve cavity extending along a direction of elongation and configured to provide fluid connectivity between the inlet port and the outlet port. The valve cavity comprises oppositely disposed linear walls in communication with the inlet port and the outlet port, and oppositely disposed lateral walls, each of the oppositely disposed lateral walls extending between the oppositely disposed linear walls. The solenoid coil is disposed in the valve body around the valve cavity. The plunger comprises a permanent magnet, and is configured to move back-and-forth along the direction of elongation between a first position and a second position in the valve cavity to selectively control the fluid connectivity between the inlet port and the outlet port, the plunger including oppositely disposed linear sides configured to face the respective oppositely disposed linear walls of the valve cavity, and adjacent curved lateral sides, each of the adjacent curved lateral sides extending between the oppositely disposed linear sides and configured to face a corresponding lateral wall of the valve cavity, the plunger having a cross sectional shape which prevents the plunger from rotating in the valve cavity, the plunger configured to fit in the valve cavity such that there are gaps between the plunger and the linear and lateral walls of the valve cavity, the gaps extending substantially the length of the direction of elongation. The controller is configured to apply at least one current to the solenoid coil to selectively move the plunger between the first position and the second position, and to selectively maintain the plunger in the first position and the second position.

### BRIEF DESCRIPTION OF THE DRAWINGS

The present disclosure will be more fully understood from the following detailed description of examples thereof, taken together with the drawings, where like numerals or characters indicate corresponding or like components. Dimensions of components and features shown in the figures are chosen for convenience and clarity of presentation and are not necessarily shown to scale. In the drawings:

FIG. 1 is a partly pictorial, partly block diagram view of a phacoemulsification system constructed and operative in accordance with an example of the present disclosure;

FIGS. 2A and 2B are views of a probe for use with the system of FIG. 1;

FIG. 3A is a schematic view of an interior of a fluid dynamics cartridge for use in the probe of FIGS. 2A and 2B;

FIG. 3B is a cross-section of the fluid dynamics cartridge through line B-B of FIG. 3A;

FIG. 3C is a cross-section of the fluid dynamics cartridge through line C-C of FIG. 3A;

FIG. 3D is a cross-section of the fluid dynamics cartridge through line D-D of FIG. 3C;

FIG. 3E-1 is a cross-section of an alternate fluid dynamics cartridge through line C-C of FIG. 3A showing a plunger in a first position;

FIG. 3E-2 is a cross-section of the alternate fluid dynamics cartridge of FIG. 3E-1 showing the plunger in second position;

FIG. 3E-3 is a cross-section of an alternate fluid dynamics cartridge through line D-D of FIG. 3C;

FIGS. 3F-1 and 3F-2 are schematic views of a shock absorber for use in the cartridge of FIG. 3C;

FIGS. 3G and 3H are cross-sectional views of alternative shock absorbers for use in the cartridge of FIG. 3C;

FIGS. 4A and 4B are schematic views of a permanent magnet in a solenoid coil;

FIGS. 5A and 5B are schematic views of operation of a solenoid valve for use in the cartridge of FIGS. 3A-3D; and

FIG. 6 is a flowchart including steps in a method of operation of the system of FIG. 1.

### DETAILED DESCRIPTION OF EXAMPLES

### System Overview

Phacoemulsification systems include an anti-vacuum-surge (AVS) cartridge at or near the proximal end of a phacoemulsification handpiece, and connects to a console, as shown, for example, in commonly owned U.S. patent application Ser. No. 17/240,505, entitled: Solenoid Valve Shock Absorber, and Ser. No. 17/511,166, entitled: Anti-Vacuum Surge Device, the disclosures of both patent applications are incorporated by reference, each in its entirety herein. The AVS cartridge includes a solenoid-type valve, with a magnetic plunger or plug, which moves within a valve cavity (also known herein as a valve chamber or bore), perpendicular to the flow path of the aspiration channel or line. The movement of the plunger is controlled by a solenoid of the valve, so that the plunger toggles between a position where the aspiration flow line is penetrated and blocked by the plunger, such that fluid and matter cannot flow through the portion of the aspiration channel within the valve cavity, and another position where the plug is beyond the aspiration flow line, where aspiration flow is not obstructed.

The plunger, in some instances, does not always form a seal with the surrounding edges of the valve cavity, resulting in some, typically negligible, leakage. Typically, the magnetic plunger is cylindrical with a circular cross section, with the valve cavity in which the plunger moves, being of a corresponding cylinder of a circular cross section with corresponding dimensions. This construction allows the plunger to be movable within the valve cavity. However, due to tolerances in geometry of the magnet, i.e., the plunger, and the valve cavity, a tight seal between the magnet and the edges of the valve chamber is difficult to obtain.

The present disclosure provides the AVS cartridge with a valve, i.e., a solenoid valve with a magnetic plunger, having parallel flattened surfaces, in a valve cavity of a rectangular or other similar cross section. The flattened surfaces of the plunger are oppositely disposed from each other and are in line with the flow path through the aspiration channel of the AVS cartridge. The cross sectional shapes of the valve cavity and plunger are less sensitive to tolerances, when compared to valve chambers/plugs of other cross sectional shapes.

### System Description

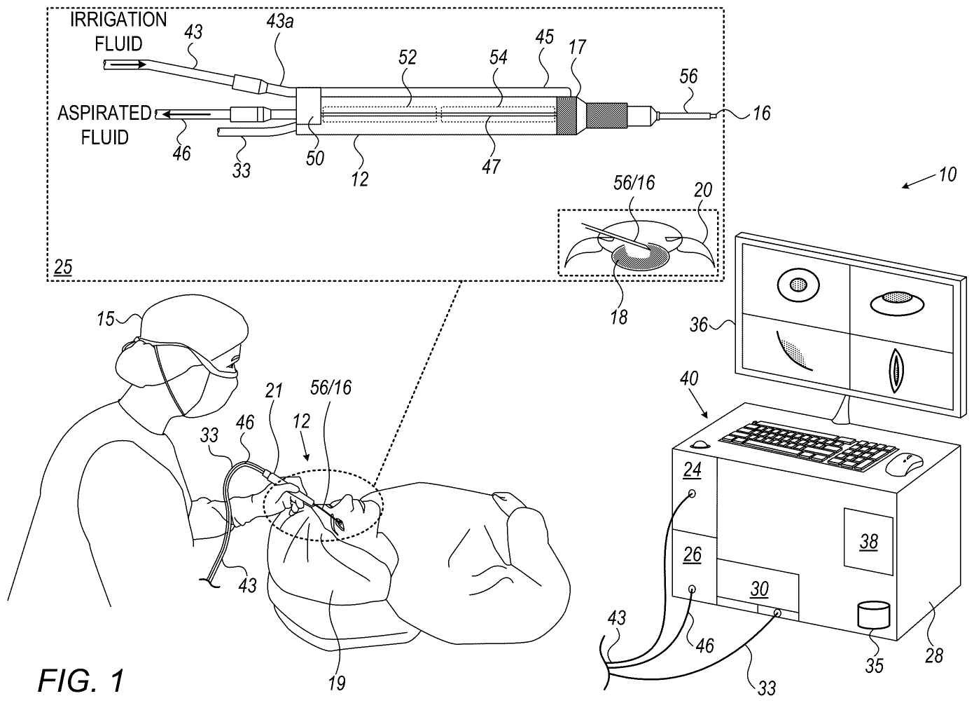

Reference is initially made to FIG. 1, which is a partial pictorial, partial block diagram, view of a phacoemulsification system 10 constructed and operative in accordance with examples of the present disclosure. When referring to the system, terms such as “upper”, “top”, “lower”, “bottom”, “upward” downward” “proximal”, “distal”, “lateral”, “vertical”, “vertically”, and their derivatives, are indicative of direction and orientation, and merely serve as examples.

The phacoemulsification system 10 includes a handheld phacoemulsification probe 12, also known as a handpiece, these terms used interchangeably herein. As seen in the pictorial view of the phacoemulsification system 10, and in the inset 25, the phacoemulsification probe 12 comprises a needle 16, a probe body 17, and a coaxial irrigation sleeve 56 that at least partially surrounds the needle 16 and creates a fluid pathway between the external wall of the needle 16 and the internal wall of the irrigation sleeve 56, where the needle 16 is hollow (hollow interior cavity) to provide an irrigation channel 45. Moreover, the irrigation sleeve 56 may have one or more side ports at, or near, the distal end to allow irrigation fluid to flow towards the distal end of the phacoemulsification probe 12 through the fluid pathway and out of the port(s).

The needle 16 is configured for insertion into a lens capsule 18 of an eye 20 of a patient 19 by a physician 15 to remove a cataract. While needle 16 (and irrigation sleeve 56) are shown in the inset 25 as a straight object, any suitable needle may be used with phacoemulsification probe 12, such as a curved or bent tip needle commercially available from Johnson & Johnson Surgical Vision, Inc., Irvine, CA, USA.

In the example of FIG. 1, during the phacoemulsification procedure, a pumping sub-system 24 comprised in a console 28 pumps irrigation fluid from an irrigation reservoir (not shown) to the irrigation sleeve 56 to irrigate the eye 20. The irrigation fluid is pumped via an irrigation tubing line 43, which runs from the console 28 to an irrigation channel 45 of the probe 12, with the distal end of the irrigation channel 45 including the fluid pathway in the irrigation sleeve 56. In another example, the pumping sub-system 24 may be coupled with or replaced by a gravity fed irrigation source such as a balanced salt solution bottle/bag.

Eye fluid and waste matter (e.g., emulsified parts of the cataract) are aspirated via an aspiration channel 47, which extends from the hollow cavity of the needle 16 through the phacoemulsification probe 12, and then via an aspiration tubing line 46 to a collection receptacle (not shown) couple with or in the console 28. The aspiration is affected, for example, by a pumping sub-system 26, which is also comprised in the console 28.

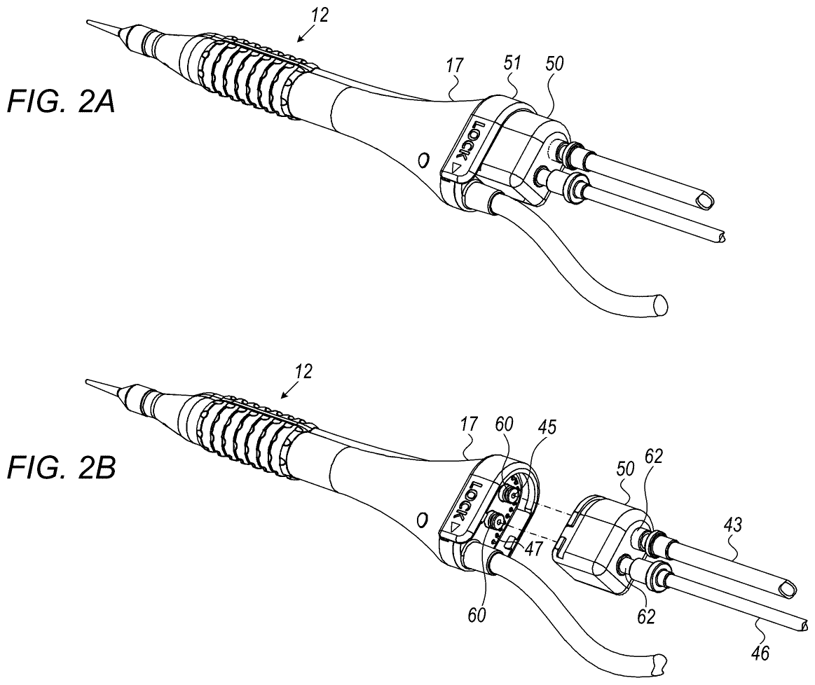

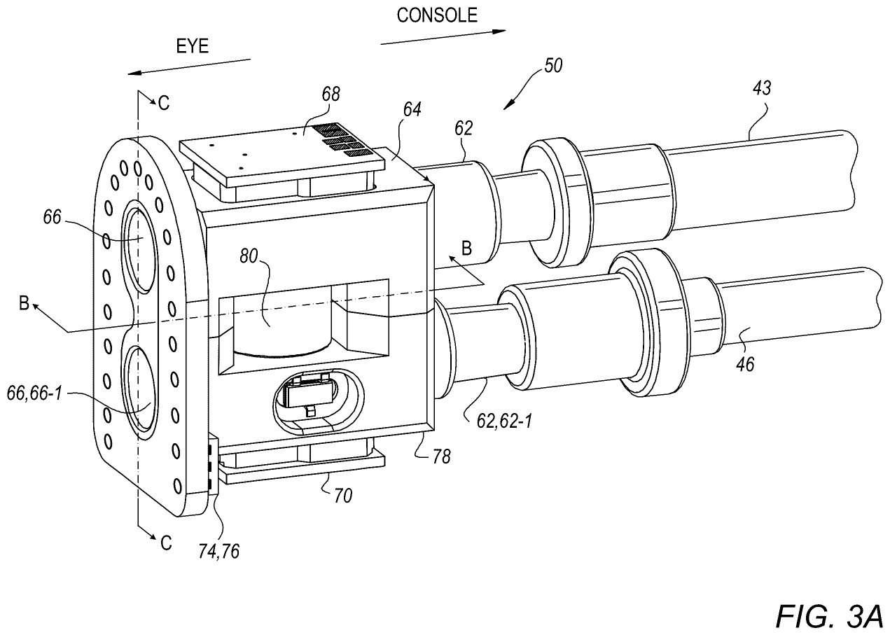

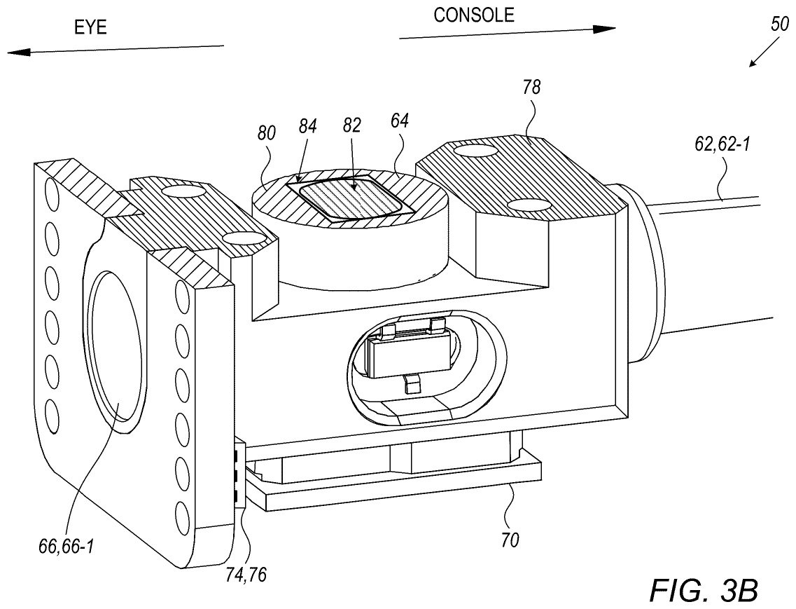

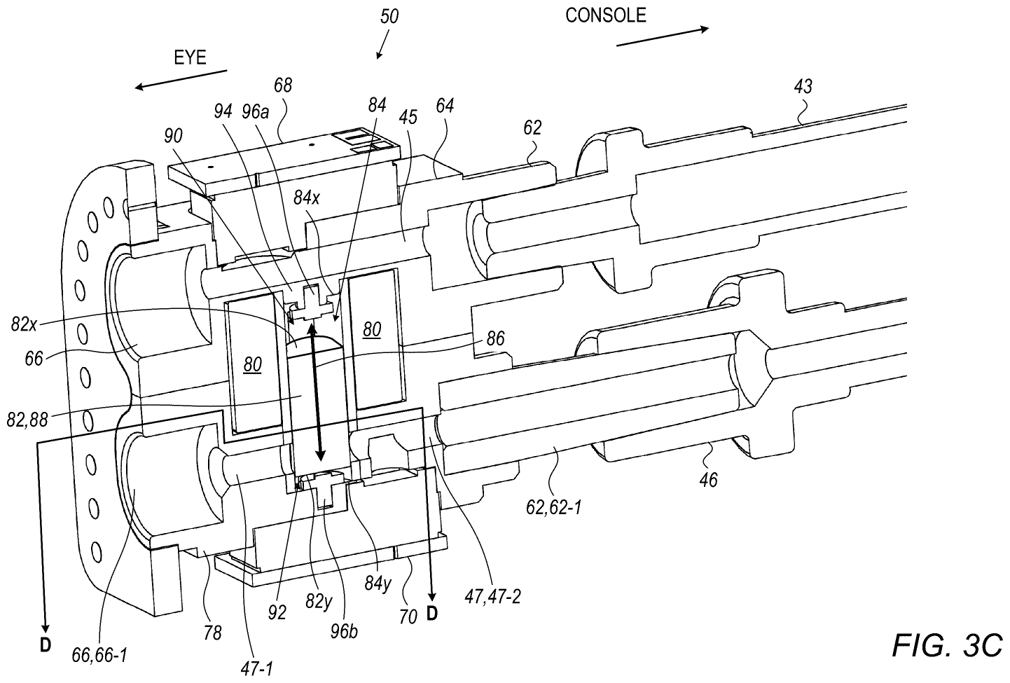

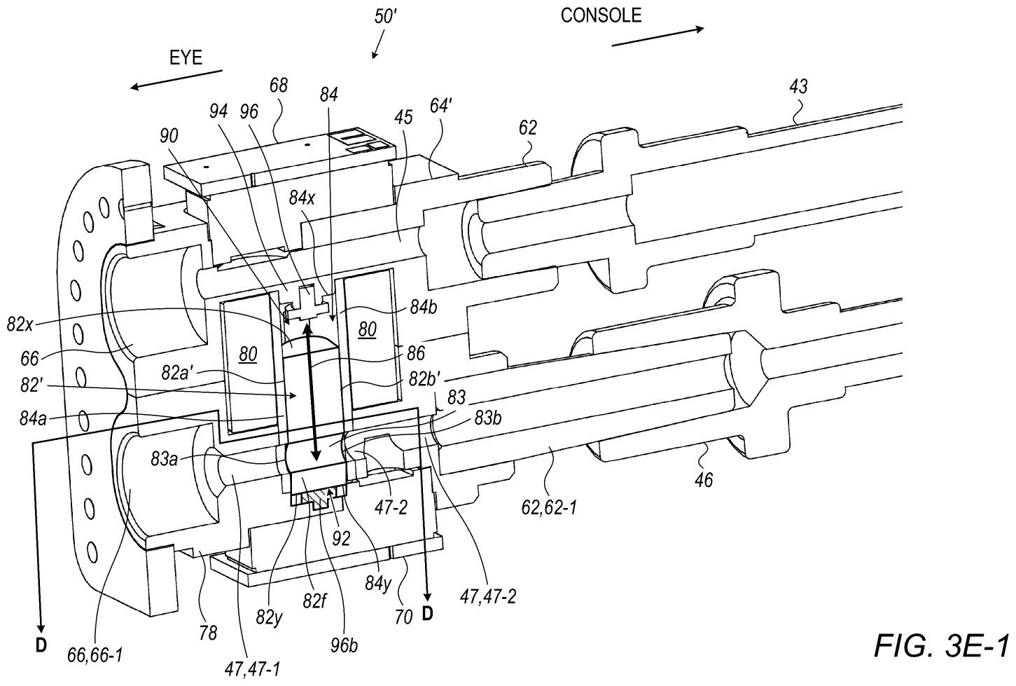

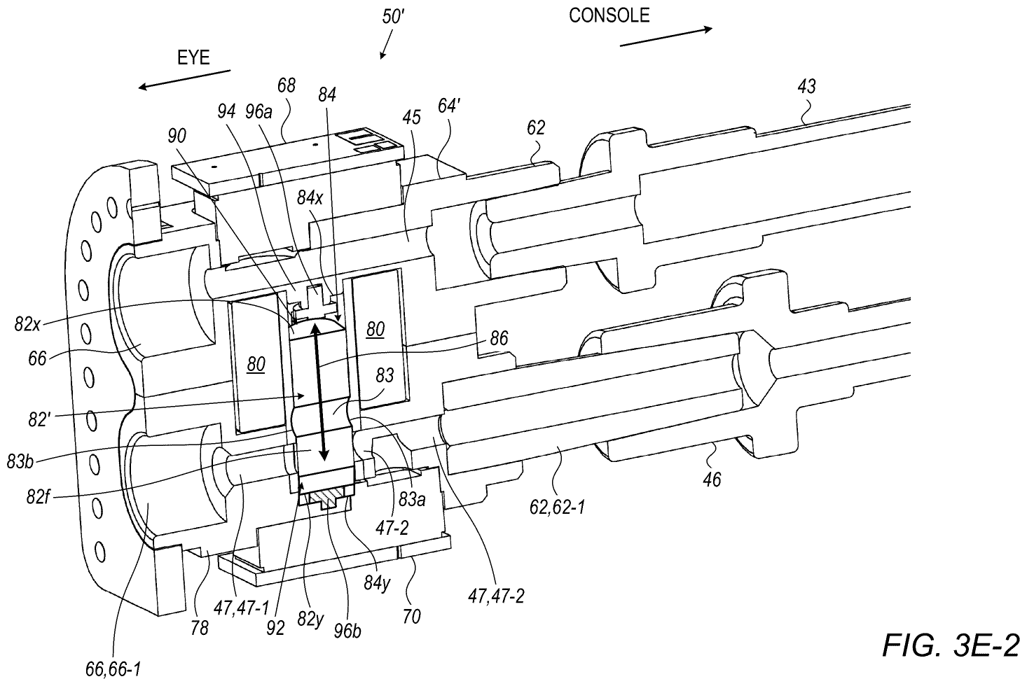

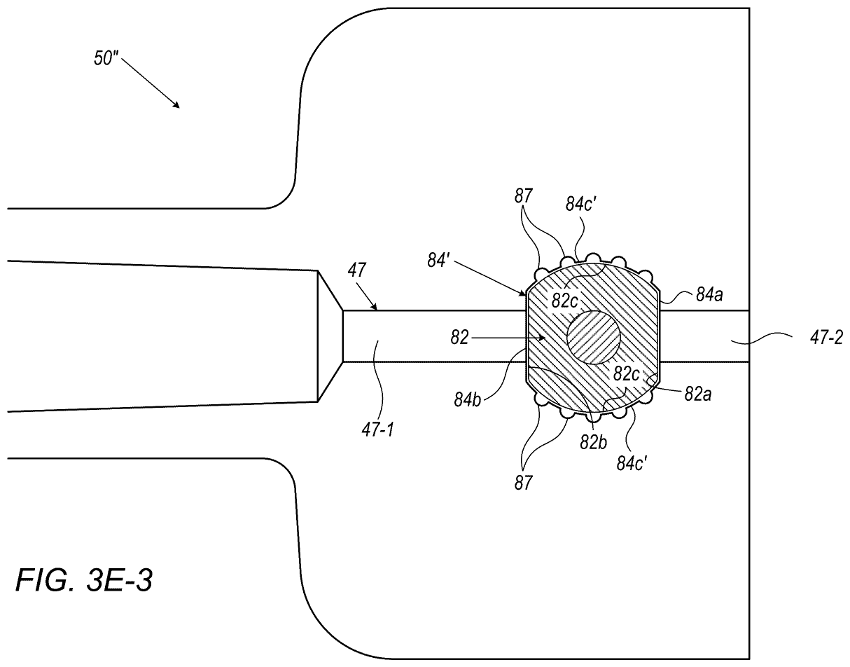

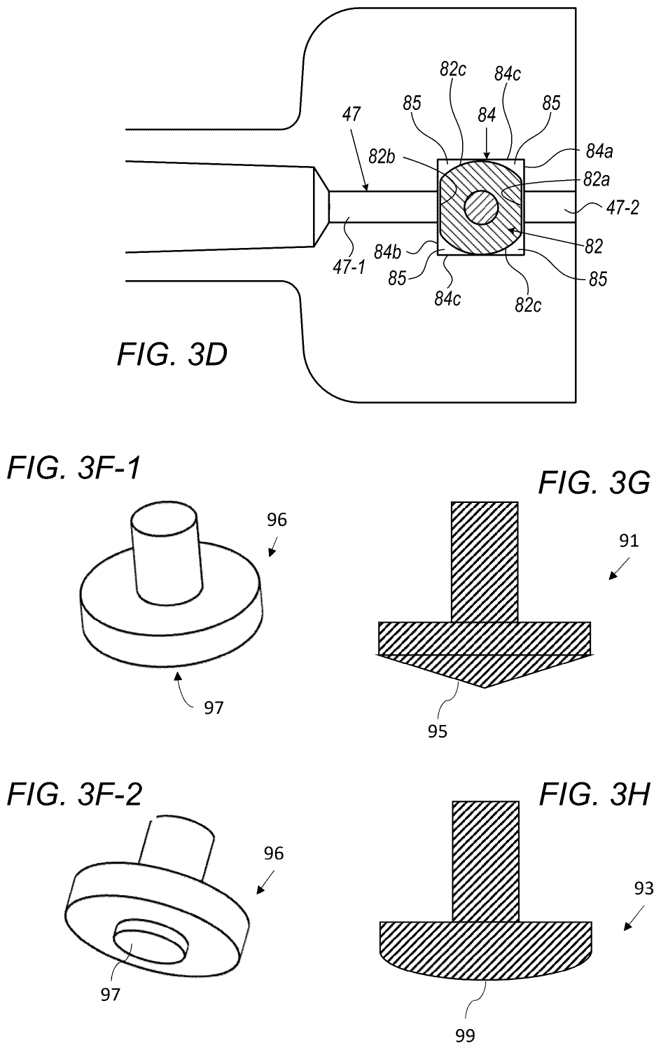

The phacoemulsification probe 12 also includes a fluid-dynamics cartridge 50. This cartridge 50 may comprise one or more valves to regulate the flow of fluid in the irrigation channel 45 and/or aspiration channel 47, as well as sensors, described in more detail with reference to FIGS. 2A, 2B and 3A-3D. For example, the aspiration tubing line 46 (of the aspiration channel 47) is typically flexible and may be prone to collapsing during an occlusion of the needle 16.

Cartridge 50 is removably couplable with probe body 17. Portions of the irrigation channel 45 and the aspiration channel 47 are disposed in the probe body 17, and portions thereof are disposed in the cartridge 50. The cartridge 50 may also be located near a proximal end of the phacoemulsification probe 12 and coupled with aspiration tubing line 46 and/or irrigation tubing line 43.

The phacoemulsification probe 12 also includes elements, such as a piezoelectric crystal(s) 52 coupled with a horn 54 to drive vibration of the needle 16. The piezoelectric crystal 52 is configured to vibrate the needle 16 in a resonant vibration mode. The vibration of the needle 16 is used to break a cataract into small pieces during a phacoemulsification procedure. The console 28 comprises a piezoelectric drive module 30, coupled with the piezoelectric crystal 52, using electrical wiring running in a cable 33. The drive module 30 is controlled by a controller 38, which conveys processor-controlled driving signals via the cable 33 to, for example, maintain the needle 16 at maximal vibration amplitude. The drive module 30 may be implemented in hardware or software, for example, in a proportional-integral-derivative (PID) control architecture. The controller 38 may also be configured to receive signals from sensors (not shown) in the phacoemulsification probe 12 and, in response to the signals, control one or more valves 64 to regulate the flow of fluid in irrigation channel 45 and/or aspiration channel 47, as described in detail below. In some examples, at least some of the functionality of the controller 38 may be implemented using a controller disposed in phacoemulsification probe 12 (e.g., in the cartridge 50).

The controller 38, for example, may receive commands via a user interface 40. Such commands may include setting a vibration mode and/or frequency of piezoelectric crystal 52 or setting or adjusting a pumping rate of pumping sub-system 24 or pumping sub-system 26. In some examples, the user interface 40 and a display 36 may be combined as a single touch screen graphical user interface. In some examples, the physician 15 uses a foot pedal (not shown) as a means of control. Additionally, or alternatively, the controller 38 may receive commands from controls located in a handle 21 of the phacoemulsification probe 12.

Some or all of the functions of the controller 38 may be combined in a single physical component or, alternatively, implemented using multiple physical components. These physical components may comprise hard-wired or programmable devices, or a combination of the two. In some examples, at least some of the functions of controller 38 may be performed by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements which are omitted for clarity of presentation. For example, the physician 15 typically performs the procedure using a stereo microscope or magnifying glasses, neither of which are shown. The physician 15 may use other surgical tools in addition to phacoemulsification probe 12, which are also not shown in order to maintain clarity and simplicity of presentation.

### The Phacoemulsification Probe

Reference is now made to FIGS. 2A and 2B, which are views of the phacoemulsification probe 12 for use with the system 10 of FIG. 1. FIG. 2A shows the cartridge 50, which is configured to be reversibly attached, to a probe body 17, for example, using a clip 51. The clip 51 is, for example, in accordance with that disclosed in commonly owned U.S. patent application Ser. No. 17/375,614, entitled: Coupling A Fluid-Dynamics Cartridge With A Phacoemulsifier Probe Body, the disclosure of which is incorporated by reference in its entirety herein. FIG. 2B shows the cartridge 50 as detached from the probe body 17. FIG. 2B shows ports 60 of the irrigation channel 45 and the aspiration channel 47 on the probe body 17 for connecting with corresponding ports (not shown in FIG. 2B, but shown in FIG. 3A) of the cartridge 50. FIG. 2B also shows the irrigation tubing line 43 and the aspiration tubing line 46 connected to ports 62 of the cartridge 50. Alternatively, cartridge 50 may be separate from the phacoemulsification probe 12 and located near the proximal end of the phacoemulsification probe 12 and coupled with the phacoemulsification probe 12 via additional tubing/connector lines (similar to those described herein) from the cartridge 50 to the phacoemulsification probe 12.

Reference is now made to FIGS. 3A-3D. FIG. 3A is a schematic view of an interior of fluid-dynamics cartridge 50, in accordance with some examples of the present disclosure, FIG. 3B is a cross-section through fluid-dynamics cartridge 50 along line B-B of FIG. 3A. FIG. 3C is a cross-section through fluid-dynamics cartridge 50 along line C-C of FIG. 3A. FIG. 3D is a cross section through fluid-dynamics cartridge 50 along line D-D of FIG. 3C.

Typically, the phacoemulsification probe 12 comprises a sensor 68, for example, a pressure sensor, and a sensor 70, for example, a vacuum sensor. The phacoemulsification probe 12 further comprises a solenoid valve 64 or valve, which is traversed by the aspiration channel 47. The sensor 68 is coupled with the irrigation channel 45. The sensor 70 is coupled with aspiration channel 47, and positioned proximally to sensor 68, as shown in FIG. 3C. Sensor 68 and sensor 70 are configured to provide respective signals (e.g., analog signals) indicative of respective fluid metrics (e.g., pressure levels) in the irrigation channel 45 and the aspiration channel 47.

The solenoid valve 64 may comprise proximal ports 62 for connection to the irrigation tubing line 43 and the aspiration tubing line 46, and distal ports 66 for connection to connection ports 60 (FIG. 2B). The inclusion of the sensors 68 and 70 in the cartridge 50 may provide higher sensitivity to local changes in fluid dynamics, and provide a higher degree of control of the pressure in the eye 20.

The phacoemulsification probe 12 may further comprise a controller 74 configured to receive the aforementioned signals from the sensor 68 and/or the sensor 70, and, responsively to the received signals, control the fluid connectivity in the irrigation channel 45 and/or the aspiration channel 47, by selectively opening and closing the solenoid valve 64. For example, the cartridge 50 may include the controller 74. The cartridge 50 may also comprise a memory 76 (e.g., an electrically erasable programmable read-only memory (EEPROM)) to store calibration settings and/or a usage counter to help avoid overusing the cartridge 50. Advantageously, the inclusion of controller 74 in the cartridge 50 may facilitate calibrating the solenoid valve 64. Additionally, or alternatively, by virtue of being relatively close to the sensors 68 and 70, the controller 74 may receive any analog signals output by these sensors 68, 70, with less added noise, relative to if the controller were disposed in console 28 (FIG. 1).

Notwithstanding the above, in some examples, the controller 74 is disposed in the console 28, or the functionality of the controller 74 is performed by the controller 38 (FIG. 1).

As shown in FIG. 3C, the aspiration channel 47 includes a section 47-1 coupled with a distal inlet port 66-1 and a section 47-2 coupled with a proximal outlet port 62-1 (as shown in FIG. 3C). The sections 47-1, 47-2 of the aspiration channel 47, include, for example, conduits extending from the respective port 66-1, 62-1 to the valve cavity 84, and as such for a path or pathway for fluid and/or matter flow via the aspiration channel 47 from the eye 20 to the console 28, through the valve cavity 84. The controller 74 is configured to control the fluid connectivity or fluid flow in the aspiration channel 47 between inlet port 66-1 and outlet port 62-1 by selectively opening and closing solenoid valve 64 responsively to the fluid metric (e.g., pressure level) in aspiration channel 47. For example, when the solenoid valve 64 is closed, the sensor 70 is configured to sense a fluid metric (e.g., pressure level) in the section 47-2 of the aspiration channel 47 between the solenoid valve 64 and the console 28.

As further shown in FIGS. 3B, 3C and 3D, the solenoid valve 64 comprises a valve body 78, a solenoid coil 80, and a plunger or plug 82, which moves within a valve cavity 84 (also known as a valve chamber or bore), for example, back and forth, e.g., up and down, along an axis of elongation 86 (e.g., oriented longitudinally), between a first position 90 and a second position 92. The valve body 78 comprises proximal ports 62 and distal ports 66 (e.g., between the inlet port 66-1 and the outlet port 62-1), which communicate with the valve cavity 84. The valve cavity 84 is configured to provide fluid connectivity between at least one pair of distal and proximal ports (e.g., between the inlet port 66-1 and the outlet port 62-1, shown in FIG. 3C) of the aspiration channel 47. A solenoid coil 80 is disposed in the valve body 78 around the valve cavity 84.

The valve cavity 84 is, for example, rectangular, including being square, in shape (e.g., cross-sectional shape), but may also be of other shapes, as disclosed below and as shown in the drawings, such as in FIG. 3E-3. The valve cavity 84 has, for example, linear-shaped (linear) walls 84a, 84b which have flat surfaces oppositely disposed from each other (see FIG. 3D). The linear walls 84a, 84b are positioned along the aspiration channel 47. The adjacent sides 84c (oppositely disposed from each other) are also, for example, linear (see FIG. 3D).

The plunger 82 includes oppositely disposed sides (i.e., linear) 82a, 82b, with flat surfaces (e.g., parallel flat surfaces defining the sides 82a, 82b), which face the corresponding (and matching) parallel flat surfaces of the correspondingly shaped walls 84a, 84b of the valve cavity 84. Oppositely disposed adjacent sides 82c of the plunger 82 are, for example, “D” shaped, e.g., curved or rounded (the curvature extending outward), and are, configured to face the oppositely disposed lateral walls 84c of the valve cavity 84. This asymmetric cross section of the plunger 82, coupled with the cross sectional shape of the valve cavity 84, prevents the plunger 82 from rotating in the valve cavity 84. Accordingly, the plunger moves linearly, e.g., vertically or longitudinally, in the valve cavity 84, along and in the direction of the axis of elongation 86.

The “D” shaped, sides 82c of the plunger 82 provide gaps 85 at corners (formed by a linear walls 84a, 84b and a corresponding adjacent lateral wall 84c) of the valve cavity 84. The gaps 85 function, for example, as vents, to allow for airflow and pressure relief in the valve cavity 84. The gaps 85 extend, for example, substantially, or completely, the length (in the longitudinal direction, e.g., the direction of the axis of elongation 86) of the valve cavity 84. The pressure relief allows the plunger 82 to move downward smoothly and evenly (for example, with the plunger lower surface 82y moving downward and upward parallel or substantially parallel to the plane of the floor 84y of the valve cavity 84). For example, when the plunger 82 moves downward, in the direction of the axis of elongation 86, the flat lower surface 82y of the plunger 82 comes to rest by seating in the valve cavity 84 firmly and flatly in abutment against the shock absorber 96b at or near the floor 84y of the valve cavity 84, to tightly seal the valve chamber 84 along the aspiration channel 47, and prevent (e.g., block) fluid and material flow through the aspiration channel 47.

The tolerances of the plunger 82 within the valve cavity 84 are such that corresponding flat surfaces of the plunger 82 sides 82a, 82b are movable along the correspondingly positioned walls 84a, 84b of the valve cavity 84. The plunger 82 moves between a first position 90, where the valve cavity 84 is open between sections 47-1, 47-2 of the aspiration channel 47, allowing for fluid connectivity (e.g., a path for fluid connectivity is open), and a second position 92 where the plunger 82 blocks the valve cavity 84 between the sections 47-1, 47-2 of the aspiration channel 47, sealing the valve cavity 84, so as to prevent fluid flow between sections 47-1, 47-2 of the aspiration channel 47 through the valve cavity 84 (e.g., block the aforementioned path for fluid connectivity).

The plunger 82, for example, is of a permanent magnet 88 (FIG. 3C) and, optionally, other components, such as a material of low friction that coats or covers the permanent magnet. In some examples, the permanent magnet may be substituted by any suitable magnetic material subjected to a force in a magnetic field, for example, but not limited to, iron, cobalt, nickel, gadolinium, and/or neodymium.

As the opening and closing of the solenoid valve 64 is performed quickly and, sometimes, many times per second, the solenoid valve 64 typically comprises one or more shock absorbers 96a, 96b, at the ceiling 84x and floor 84y of the valve cavity 84, as shown in FIG. 3C. As also shown in FIGS. 3F-1 and 3F-2, the respective shock absorbers 96a, 96b, collectively element 96 in FIGS. 3F-1 and 3F-2, are oriented such that a flat surface 97 thereof faces the plunger 82. Each shock absorber 96a, 96b serves to soften the striking of the plunger 82 against the valve body 78 along the axis of elongation 86, reducing wear and tear on the valve 64. The shock absorbers 96a, 96b, for example, are generally formed from a resilient material such as silicone rubber, natural rubber, synthetic rubber, or polyurethane. As shown in FIG. 3C, the upper shock absorber 96a may be disposed within a spacer 94.

FIGS. 3G and 3H show cross-sectional views of alternative shock absorbers 91, 93 for use in the cartridge 50, in the valve cavity 84. Two shock absorbers 91 or two shock absorber 93 (or any suitable combination of one of each of the shock absorbers 91, 93) may be used in the solenoid valve 64 instead of the shock absorbers 96a, 96b. The shock absorber 91 of FIG. 3G includes a conical surface 95, which faces the plunger 82. The shock absorber 93 of FIG. 3H includes a rounded surface 99, which faces plunger 82.

The controller 74 (FIGS. 3A and 3B) is configured to apply at least one current to the solenoid coil 80. The current applied allows for the selective movement of the plunger 82, between a first position 90 and a second position 92, and, to selectively maintain the plunger in first position 90 or second position 92.

FIGS. 3E-1 and 3E-2 show an alternate example of a cartridge 50′ showing a solenoid valve 64′, with all elements numbered similarly to those of cartridge 50 and solenoid valve 64, except the plunger 82′ is different from the plunger 82 of FIG. 3C. The plunger 82′ includes a tubular bore (tube or segment) 83, extending transversely (e.g., perpendicular, or substantially perpendicular to the axis of elongation 86) through the plunger 82′.

The tubular bore 83 is, for example, circular or rounded in cross section, and typically of a diameter approximately or the same as the diameter of the aspiration sections 47-1 and 47-2, and open at its ends 83a, 83b (at the edges of the sides 82a′, 82b′ of the plunger 82′). The tubular portion 83 is configured to be in a first or open position, shown in FIG. 3E-1, where the tubular bore 83 aligns with the aspiration channel 47 (e.g., conduits of sections 47-1 and 47-2), providing an open pathway for fluid and matter to flow from aspiration channel 47 section 47-1 to aspiration channel 47 section 47-2 (from the eye 20 to the console 28). To close the aspiration channel 47 and obstruct flow therethrough, the plunger 82′ is moved upward, into the position, e.g., a second or closed (blocking) position, shown in FIG. 3E-2, such that a foot portion 82f moves into the aspiration channel 47, blocking or otherwise occluding fluid and matter flow therethrough. The movement of the plunger 82′ is in accordance with the description provided herein for the plunger 82 of the solenoid valve 64 of the cartridge 50.

FIG. 3E-3 shows an alternate example of a cartridge 50″, with all elements numbered similarly to those of cartridge 50 and solenoid valve 64, except the valve cavity 84′ is different from the valve cavity 84 in that it includes curved lateral walls 84c′ corresponding to the curvature of the lateral sides of the 82c of the plunger 82, and grooves 87 extending outward (in a concave manner), into the walls 84c′ of the valve cavity 84′. The remainder of the valve cavity 84′ is similar to the valve cavity 84, detailed above.

The grooves 87 extend, for example, longitudinally, in the direction of the axis of elongation 86, and including extending the complete length (or substantially the complete length), of the valve cavity 84′. The grooves 87 are semicircular or rounded in shape, but may be other shapes such as rectangular, triangular, oval, and the like. Similar to the gaps 85 in the valve cavity 84, the grooves 87 function as vents, to allow for airflow and pressure relief in the valve cavity 84′. The pressure relief allows the plunger 82 to move downward smoothly and evenly (for example, with the lower surface 82y (FIG. 3C) moving downward and upward parallel or substantially parallel to the plane of the floor 84y of the valve cavity 84), such that the plunger 82 seats flatly and evenly on the shock absorber 96b proximate to or at the floor 84y (FIG. 3C) of the valve cavity 84′. The movement of the plunger 82 in the valve cavity 84′ of the solenoid valve 64 and cartridge 50″ is in accordance with the description provided herein for the plunger 82 of the solenoid valve 64 of the cartridge 50.

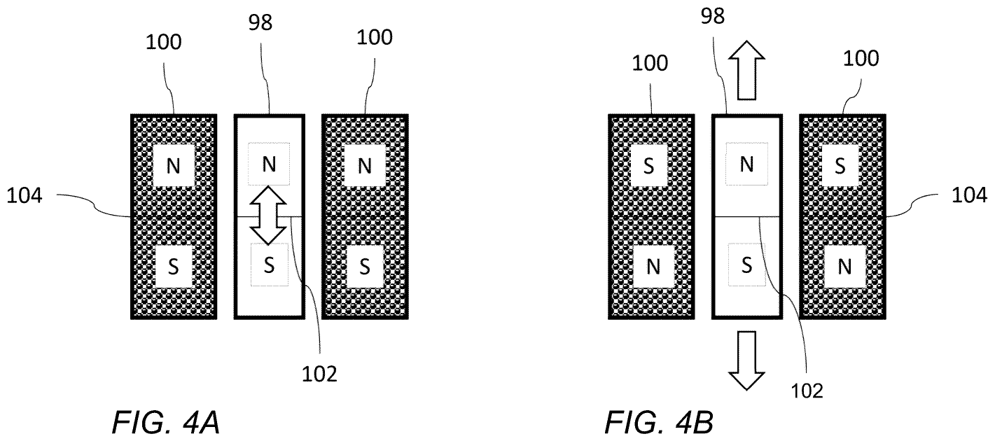

Reference is now made to FIGS. 4A and 4B, which are schematic views of a permanent magnet 98 (representative of the plunger 82) in a solenoid coil 100 (representative of solenoid coil 80). As magnetics are involved the polarity of the magnetic structures is indicated by “N” for one polarity or North, and by “S” for the other and opposite polarity or South.

In the configuration of FIG. 4A, the polarity of the solenoid coil 100 is in the same direction as the polarity of the permanent magnet 98. In such a configuration, if a center 102 of the permanent magnet 98 is moved slightly away from a center 104 of the solenoid coil 100, the permanent magnet 98 will oscillate around the center 104 of the solenoid coil 100 until the permanent magnet 98 settles, so that the center 102 of the permanent magnet 98 is aligned with the center 104 of the solenoid coil 100. The permanent magnet 98 therefore rests in a stable position with respect to the solenoid coil 100.

In the configuration of FIG. 4B, the polarity of the solenoid coil 100 is in the opposite direction to the polarity of the permanent magnet 98. In such a configuration, if the center 102 of the permanent magnet 98 is moved slightly away from the center 104 of the solenoid coil 100, the permanent magnet 98 will continue to move in that direction. The permanent magnet 98 in FIG. 4B is therefore in an unstable position with respect to the solenoid coil 100.

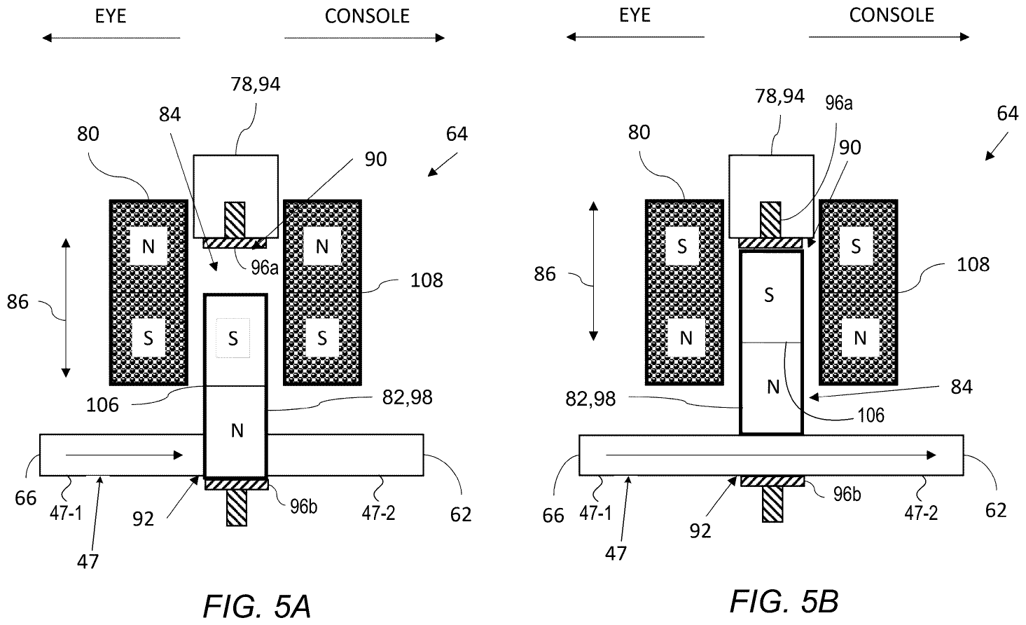

Reference is now made to FIGS. 5A and 5B, which are schematic views of operation of the solenoid valve 64 for use in the cartridge 50 of FIGS. 3A to 3D.

The plunger 82 is configured to move back-and-forth along the direction of the axis of elongation 86 between position 90 (for example, a first position where the aspiration channel 47 is not blocked) and position 92 (for example, a second position where the aspiration channel 47 is blocked or otherwise obstructed) and in the valve cavity 84. This plunger 82 movement selectively controls the fluid connectivity between respective ones of the ports 66, 62 (and the sections 47-1, 47-2 of the aspiration channel 47). The controller 74 (FIGS. 3A and 3B) is configured to apply current to the solenoid coil 80 to selectively move the plunger 82 between the position 92 and position 90, and to selectively maintain the plunger 82 in the respective positions 90, 92. FIG. 5A shows the plunger 82 in the position 92 blocking fluid connectivity in the aspiration channel 47. FIG. 5B shows the plunger 82 in the position 90, allowing fluid connectivity in the aspiration channel 47.

The plunger 82 does not have a fixed rest position in the valve cavity 84. Even though in some orientations the plunger 82 may fall in one of the positions 90, 92 due to gravity, if the solenoid valve 64 is oriented differently, the plunger 82 may fall to a different position. The plunger 82 does not include a restoring element (e.g., spring) configured to restore the plunger 82 to a fixed rest position. The plunger 82 will not always remain in the position 92 or position 90 (e.g., if the orientation of the phacoemulsification probe 12 is changed) without applying current to the solenoid coil 80. In other words, for the solenoid valve 64 to function correctly, a current is applied to the solenoid coil 80 whether the solenoid valve 64 is to remain open or closed. The plunger 82 will remain in the position 90 or the position 92, upon application of current (e.g., electric current) to the solenoid coil 80.

The controller 74 is configured to apply a current to the solenoid coil 80 to activate the solenoid coil 80 with a polarity to cause the plunger 82 to move and be maintained in the position 92 as shown in FIG. 5A (aspiration channel 47 is closed or blocked with the plunger abutting the shock absorber 96a). The controller 74 is configured to apply an opposite current to the solenoid coil 80 to activate the solenoid coil 80 with an opposite polarity to cause the plunger 82 to move and be maintained in the position 90 as shown in FIG. 5B (aspiration channel 47 is open).

The permanent magnet 98 of the plunger has a center 106 with respect to the direction of the axis of elongation 86. The solenoid coil 80 has a center 108 with respect to the direction of axis of elongation 86.

The valve body 78 includes the spacer 94, to prevent the center 106 of the magnet (permanent magnet 98 of the plunger 82) moving in the direction of the axis of elongation 86 past the center 108 of the solenoid coil 80. Therefore, the spacer 94 maintains asymmetry between the center 108 of the solenoid coil 80 and the center 106 of the permanent magnet 98 of the plunger 82 with respect to the direction of the axis of elongation 86, so that the centers 106, 108 are, for example, never aligned with respect to the direction of the axis of elongation 86. The above asymmetry is desirable to allow movement of the permanent magnet 98 (the plunger 82) within the valve cavity 84 to be controlled, and the position of the permanent magnet 98 (of the plunger 82) is maintained at the position 90, such that the plunger 82 is stable (as explained above with reference to FIGS. 4A and 4B). For example, when the plunger 82 is in the position 90, the plunger 82 abuts the spacer 94 via abutting the shock absorber 96a (see FIG. 5B).

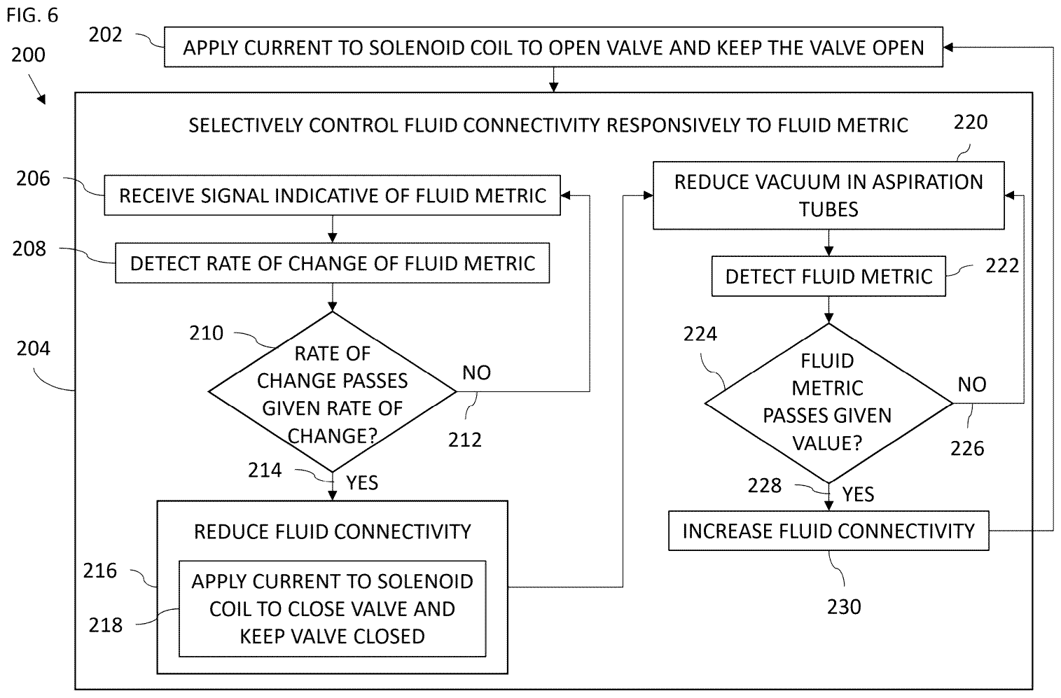

Reference is now made to FIG. 6, which is a flowchart 200. The flowchart 200 includes steps (processes and/or subprocesses) in an exemplary method of operation of the system 10 of FIG. 1. Reference is also made to FIGS. 3C and 3D.

The controller 74 is configured to apply (block 202) a current to the solenoid coil 80 to activate the solenoid coil 80 with a polarity to cause the plunger 82 to move and be maintained in the position 90 so that the solenoid valve 64 is open (and kept open) and there is fluid connectivity along the aspiration channel 47.

The controller 74 is configured to selectively control (block 204) the fluid connectivity responsively to a measured fluid metric (e.g., a sensed fluid flow or pressure level) in the phacoemulsification probe 12. In some examples, the controller 74 is configured to selectively control the fluid connectivity responsively to the fluid metric from the one or more sensors 68, 70 coupled with the aspiration channel 47. In this example, the sensor(s) 68, 70 detect a change in pressure. The step of block 204 is now described in more detail with reference to sub-steps of blocks 206-230.

The controller 74 is configured to receive a signal indicative of the fluid metric (e.g., pressure level) in the aspiration channel 47 from the sensor 70 (block 206). The controller 74 is configured to detect a rate of change of the fluid metric (e.g., pressure level) in the aspiration channel 47 responsively to the received signal (block 208). At a decision block 210, the controller 74 is configured to determine whether the rate of change passes (e.g., exceeds) a given rate of change. If the rate of change does not pass (e.g., exceed) the given rate of change (branch 212), the method returns to the sub-step of block 206. However, if the rate of change passes (e.g., exceeds) the given rate of change (branch 214), the controller 74 is configured to reduce the fluid connectivity (block 216) between the inlet port 66-1 and the outlet port 62-1. The sub-step of block 216 may include the controller 74 being configured to apply a current to the solenoid coil 80 to activate the solenoid coil 80 with an opposite polarity to cause the plunger 82 to move and be maintained in the position 92 (block 218). The solenoid valve 64 is closed and kept closed thereby blocking fluid connectivity in the aspiration channel 47 at the location of the plunger 82 at the location of thereby isolating the eye 20 from the aspiration tubing line 46 (FIG. 1) and protecting the eye 20 from a vacuum surge.

In some examples, rather than the solenoid valve 64 closing completely and fast, the solenoid valve 64 may be controlled to close partially and/or slowly. In some examples, the activation of the solenoid valve 64 may also be controlled according to pressure, flow, temperature, or a combination of these type of sensed parameters.

The controller 74 is configured to reduce the vacuum in the aspiration tubing line 46 (block 220) (and the portion of the aspiration channel 47 between the solenoid valve 64 and the aspiration tubing line 46), for example, by reducing the action of the pumping sub-system 26, or opening a vent in the aspiration tubing line 46 or in the aspiration channel 47. The controller 74 is configured to detect the fluid metric (e.g., the pressure level) in the aspiration channel 47 responsively to signal received from the sensor 70 (block 222). At a decision block 224, the controller 74 is configured to determine if the fluid metric (e.g., pressure level) passes (e.g., exceeds) a given value (e.g., given pressure level). If the fluid metric (e.g., pressure level) does not pass (e.g., exceed) the given value (e.g., given pressure level) (branch 226), the sub-step of block 220 is repeated. If the fluid metric (e.g., pressure level) passes (e.g., exceeds) the given value (e.g., given pressure level) (branch 228), the controller 74 is configured to increase (block 230) the fluid connectivity between the inlet port 66-1 and the outlet port 62-1 responsively to the fluid metric (e.g., pressure level) passing (e.g., exceeding) a given value (e.g., a given pressure level), for example, the step of block 202 is repeated.

### EXAMPLES

### Example 1

An ophthalmic fluid dynamics system, which comprises a solenoid valve. The solenoid valve comprises: a valve body, a solenoid coil, a plunger and a controller. The valve body comprises ports including an inlet port and an outlet port, and a valve cavity extending along a direction of elongation and configured to provide fluid connectivity between the inlet port and the outlet port. The valve cavity comprises oppositely disposed linear walls in communication with the inlet port and the outlet port, and oppositely disposed lateral walls, each of the oppositely disposed lateral walls extending between (and adjacent to) the oppositely disposed linear walls. The solenoid coil is disposed in the valve body around the valve cavity. The plunger comprises a permanent magnet, and is configured to move back-and-forth along the direction of elongation between a first position and a second position in the valve cavity to selectively control the fluid connectivity between the inlet port and the outlet port, the plunger including oppositely disposed linear sides configured to face the respective oppositely disposed linear walls of the valve cavity, and adjacent curved lateral sides, each of the adjacent curved lateral sides extending between the oppositely disposed linear sides and configured to face a corresponding lateral wall of the valve cavity, the plunger having a cross sectional shape which prevents the plunger from rotating in the valve cavity, the plunger configured to fit in the valve cavity such that there are gaps between the plunger and the linear walls and the lateral walls of the valve cavity, the gaps extending substantially or the entire length of the direction of elongation in the valve cavity. The controller is configured to apply at least one current to the solenoid coil to selectively move the plunger between the first position and the second position, and to selectively maintain the plunger in the first position and the second position.

### Example 2

The system of Example 1, such that the plunger does not have a fixed rest position in the valve cavity.

### Example 3

The system of Example 1 or Example 2, such that the plunger does not include a restoring element configured to restore the plunger to a fixed rest position.

### Example 4

The system of any one of Example 1 to Example 3, such that the plunger will not remain in the first position and second position without applying the at least one current to the solenoid coil.

### Example 5

The system of any one of Example 1 to Example 4, such that the plunger will remain in the first position or the second position upon application of the at least one current to the solenoid coil.

### Example 6

The system of any one of Example 1 to Example 5, such that the plunger additionally comprises a bore through the plunger and oriented substantially perpendicular to the direction of elongation, such that when the plunger is in the first position, the bore providing a path for fluid connectivity between the respective ones of the ports, and when the plunger is in the second position, the plunger blocks the path for fluid connectivity between the inlet port and the outlet port.

### Example 7

The system of any one of Example 1 to Example 6, such that the plunger is in the first position the bore aligns with conduits into the valve cavity from the inlet port and the outlet port.

### Example 8

The system of any one of Example 1 to Example 7, such that each of the oppositely disposed lateral walls of the valve cavity are linear.

### Example 9

The system of any one of Example 1 to Example 8, wherein the valve cavity is rectangular in cross-sectional shape.

### Example 10

The system of any one of Example 1 to Example 9, such that each of the oppositely disposed lateral walls of the valve cavity are curved.

### Example 11

The system of any one of Example 1 to Example 10, such that each of the oppositely disposed lateral walls of the valve cavity are curved correspondingly to the adjacent curved lateral sides of the plunger.

### Example 12

The system of any one of Example 1 to Example 11, such that each of the oppositely disposed lateral walls of the valve cavity include one or more grooves extending into each of the lateral walls.

### Example 13

The system of any one of Example 1 to Example 12, such that the controller is configured to: apply a first current to the solenoid coil to activate the solenoid coil with a first polarity to cause the plunger to move and be maintained in the first position; and, apply a second current to the solenoid coil to activate the solenoid coil with a second opposite polarity to cause the plunger to move and be maintained in the second position.

### Example 14

The system of any one of Example 1 to Example 13, such that the solenoid valve includes an irrigation channel extending therethrough, and an aspiration channel extending through the valve cavity, between the inlet port and the outlet port.

### Example 15

The system of any one of Example 1 to Example 14, additionally comprising a medical tool for communicating with the solenoid valve. The medical tool comprises: a body; a needle mounted in the body and configured for being vibrated, the needle including a cavity extending therethrough; and, the body including an irrigation channel, and an aspiration channel, extending from the cavity of the needle through the body, wherein when the medical tool is in communication with the solenoid valve, the aspiration channel of the medical tool communicates with the aspiration channel of the solenoid valve, and the irrigation channel of the medical tool communicates with the irrigation channel of the solenoid valve.

### Example 16

The system of any one of Example 1 to Example 15, such that the solenoid valve additionally comprises: a sensor configured to provide a signal indicative of a fluid metric in the aspiration channel, the controller being configured to selectively control the fluid connectivity in the aspiration channel between the inlet port and the outlet port responsively to the fluid metric.

### Example 17

The system of any one of Example 1 to Example 16, such that the fluid metric is a pressure level.

### Example 18

The system of any one of Example 1 to Example 17, such that the controller is configured to detect a rate of change of the fluid metric in the aspiration channel, and reduce the fluid connectivity between the inlet port and the outlet port responsively to the detected rate of change passing a given rate of change.

### Example 19

The system of any one of Example 1 to Example 18, such that the controller is configured to increase the fluid connectivity between the inlet port and the outlet port responsively to the fluid metric passing a given value.

### Example 20

The system of any one of Example 1 to Example 19, such that the first position of the plunger is such that a path for fluid connectivity is open between the inlet port and the outlet port in the valve cavity, and the second position of the plunger is such that the path for fluid connectivity is blocked in the valve cavity.

As used herein, the terms “about” or “approximately” for any numerical values or ranges indicate a suitable dimensional tolerance that allows the part or collection of components to function for its intended purpose as described herein.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.

The examples described of the present disclosure are not limited by what has been particularly shown and described hereinabove. Rather the scope of the disclosure includes both combinations and sub-combinations of the various features described hereinabove, as well as variations and modifications thereof which would occur to persons skilled in the art upon reading the foregoing description and which are not disclosed in the prior art.

1. An ophthalmic fluid dynamics system, comprising:
a solenoid valve comprising:
a valve body comprising ports including an inlet port and an outlet port, a valve cavity extending along a direction of elongation and configured to provide fluid connectivity between the inlet port and the outlet port, the valve cavity comprising oppositely disposed linear walls in communication with the inlet port and the outlet port, and oppositely disposed lateral walls, each of the oppositely disposed lateral walls extending between the oppositely disposed linear walls;
a solenoid coil disposed in the valve body around the valve cavity;
a plunger comprising a permanent magnet, and configured to move back-and-forth along the direction of elongation between a first position and a second position in the valve cavity to selectively control the fluid connectivity between the inlet port and the outlet port, the plunger including oppositely disposed linear sides configured to face the respective oppositely disposed linear walls of the valve cavity, and adjacent curved lateral sides, each of the adjacent curved lateral sides extending between the oppositely disposed linear sides and configured to face a corresponding lateral wall of the valve cavity, the plunger having a cross sectional shape which prevents the plunger from rotating in the valve cavity, the plunger configured to fit in the valve cavity such that there are gaps between the plunger and the linear and lateral walls of the valve cavity, the gaps extending substantially the length of the direction of elongation; and
a controller configured to apply at least one current to the solenoid coil to selectively move the plunger between the first position and the second position, and to selectively maintain the plunger in the first position and the second position.

2. The system according to claim 1, wherein the plunger does not have a fixed rest position in the valve cavity.

3. The system according to claim 1, wherein the plunger does not include a restoring element configured to restore the plunger to a fixed rest position.

4. The system according to claim 1, wherein the plunger will not remain in the first position and second position without applying the at least one current to the solenoid coil.

5. The system according to claim 1, wherein the plunger will remain in the first position or the second position upon application of the at least one current to the solenoid coil.

6. The system according to claim 1, wherein the plunger additionally comprises a bore through the plunger and oriented substantially perpendicular to the direction of elongation, such that when the plunger is in the first position, the bore providing a path for fluid connectivity between the respective ones of the ports, and when the plunger is in the second position, the plunger blocks the path for fluid connectivity between the inlet port and the outlet port.

7. The system according to claim 6, wherein when the plunger is in the first position the bore aligns with conduits into the valve cavity from the inlet port and the outlet port.

8. The system according to claim 1, wherein each of the oppositely disposed lateral walls of the valve cavity are linear.

9. The system according to claim 8, wherein the valve cavity is rectangular in cross-sectional shape.

10. The system according to claim 1, wherein each of the oppositely disposed lateral walls of the valve cavity are curved.

11. The system according to claim 10, wherein each of the oppositely disposed lateral walls of the valve cavity are curved correspondingly to the adjacent curved lateral sides of the plunger.

12. The system according to claim 11, wherein each of the oppositely disposed lateral walls of the valve cavity include one or more grooves extending into each of the lateral walls.

13. The system according to claim 1, wherein the controller is configured to:
apply a first current to the solenoid coil to activate the solenoid coil with a first polarity to cause the plunger to move and be maintained in the first position; and
apply a second current to the solenoid coil to activate the solenoid coil with a second opposite polarity to cause the plunger to move and be maintained in the second position.

14. The system according to claim 1, wherein the solenoid valve includes an irrigation channel extending therethrough, and an aspiration channel extending through the valve cavity, between the inlet port and the outlet port.

15. The system according to claim 14, additionally comprising:
a medical tool for communicating with the solenoid valve, comprising:
a body;
a needle mounted in the body and configured for being vibrated, the needle including a cavity extending therethrough; and
the body including an irrigation channel, and an aspiration channel, extending from the cavity of the needle through the body,
wherein when the medical tool is in communication with the solenoid valve, the aspiration channel of the medical tool communicates with the aspiration channel of the solenoid valve, and the irrigation channel of the medical tool communicates with the irrigation channel of the solenoid valve.

16. The system according to claim 14, wherein the solenoid valve additionally comprises:
a sensor configured to provide a signal indicative of a fluid metric in the aspiration channel, the controller being configured to selectively control the fluid connectivity in the aspiration channel between the inlet port and the outlet port responsively to the fluid metric.

17. The system according to claim 16, wherein the fluid metric is a pressure level.

18. The system according to claim 16, wherein the controller is configured to detect a rate of change of the fluid metric in the aspiration channel, and reduce the fluid connectivity between the inlet port and the outlet port responsively to the detected rate of change passing a given rate of change.

19. The system according to claim 16, wherein the controller is configured to increase the fluid connectivity between the inlet port and the outlet port responsively to the fluid metric passing a given value.

20. The system according to claim 1, wherein the first position of the plunger is such that a path for fluid connectivity is open between the inlet port and the outlet port in the valve cavity, and the second position of the plunger is such that the path for fluid connectivity is blocked in the valve cavity.
